(19) United States
(12) Patent Application Publication
(10) Pub. No.: US 2006/0003016 A1
(43) Pub. Date: Jan. 5, 2006

(54) Therapeutic method for endolymphatic hydrops associated diseases

(76) Inventors: Hideaki Naganuma, Tokyo (JP); Katsumasa Kawahara, Tokyo (JP)

Correspondence Address: YOUNG & THOMPSON, 745 SOUTH 23RD STREET, 2ND FLOOR, ARLINGTON, VA, 22202, US

(21) Appl. No.: 10/879,166

(22) Filed: Jun. 30, 2004

(51) Int. Cl. A61K 33/00

(52) U.S. Cl. 424/600

(57) ABSTRACT

A therapeutic method completely opposite to the known basic therapeutic policy including restriction of water and salt intake, which has been hitherto designated as the therapeutic method for Meniere's disease, and is a therapeutic method for endolymphatic hydrops associated diseases by performing water intake therapy for the patient with strongly suspected Meniere's disease from the initial stage of disease. For preventing vertiginous attack and hearing loss by performing patient compliance instruction for proper intake of water and maintaining plasma AVP at low level: sufficient water intake and short term administration of osmotic diuretic in the patient are performed; plasma osmotic pressure is maintained at the lowest limit of the normal level; secretion of AVP from the pituitary grand is minimized; proper activity of AQP2 in the inner ear is maintained; influent of water into the endolymph is decreased; and prevention of vertiginous attack and improvement of hearing are performed.

### TECHNICAL FIELDS

The present invention relates to a novel therapeutic method for Meniere's disease and endolymphatic hydrops associated diseases.

### BACKGROUND ART

Meniere's disease, originally named by Dr. Prosper Meniere, is a clinical disorder of the inner ear characterized by vertigo, hearing loss, and tinnitus. According to histopathologic study of the temporal bone after patients' death, the underlying pathophysiologic state in Meniere's disease is endolymphatic hydrops. Endolymphatic hydrops is a condition of excess endolymph in the inner ear and the endolymphatic space. Endolymphatic hydrops in the cochlea and vestibule, respectively, causes hearing loss and vertigo. However, a mechanism of pathogenesis is unknown.

Besides medication during attacks and the psychologic support during attack-free interval, the most popular and standard treatment for patients with Meniere's disease is osmotic diuretics (isosorbide) or hyperosmolality. Further, “low salt and low water” in a daily life is recommended as a golden rule¹³⁾, since Harrison and Naftalin¹⁹⁾ described that acute Meniere's attack occurred when the level of aldosterone secretion was low and water was gained by kidney due to an excess of plasma vasopressin (AVP: arginine vasopressin, a peptide hormone secreted from posterior lobe of the pituitary gland in response to plasma osmolality). On the other hand, Comacchio et al. (1992) reported that Meniere's disease in congenital nephrogenic diabetes insipidus is improved when plasma AVP level is decreased. This indicates that a direct pathological incidence is not (gained) water itself, and hints us that a high level in plasma AVP may cause Meniere's disease.

Recently, development of molecular biological techniques has revealed an existence of several isoforms of water channels (AQP1-AQP6)¹⁾⁻¹⁰⁾ and ¹⁶⁾¹⁷⁾ and Na+-K+-2Cl cotransporter (NKCC2) in the inner ear. These molecules, being related to the production of endolymph, may be responsible for endolymphatic hydrops. Interestingly, Takeda et al.¹²⁾ reported that the level of plasma AVP was significantly higher in patients with Meniere's disease (This result is now in contradiction to the results of others who reported no significant increase compared with control²⁰⁾. Further, Takeda et al.¹¹⁾ demonstrated that in guinea pig endolymphatic hydrops was produced after administration of AVP. In addition, we also showed an increase in ABR (auditory brainstem evoked response) threshold in arginine-vasopressin induced rats¹⁸⁾. All these results strongly suggest that AVP is likely to be responsible for Meniere's disease.

### PROBLEMS IN THE PRESENT TREATMENT

The standard treatment for patients with Meniere's disease starts from the microscopic examination of endolymphatic hydrops and the hypothesis by Harrison and Naftalin¹⁹⁾ who described Meniere's disease as a disturbance of the control of both salt and water balances. To reduce production of a surplus endolymph, “salt and water restriction” was recommended in combination with osmotic diuretics¹³⁾. This treatment, hyperosmotic therapy, improves acute symptoms of Meniere's disease probably due to decrease in endolymphatic pressure. However, more than half of the patients are re-attacked and their hearing ability worsen despite the standard treatment. In these cases, more AVP may be secreted in response to the hyperosmotic therapy.

### DISCLOSURE OF THE INVENTION

Based on our long-term experience on clinical treatment for patients with Meniere's disease, we have invented a new treatment: water intake method for patients with Meniere's disease.

Therapeutic method of our invention is always performed after general examinations and standard laboratory examinations, such as blood pressure, plasma electrolytes, and urine osmolality. After consideration of body weight, age, sex, and life style, we directed the patients to take adequate water (see below) every day in combination with short-term application with isosorbite®. During the initial stage of the water intake method, recommended volume of water intake for adult male, weighing 60 kg, is calculated as 1.8-2.1 l per day according to the following formula (F 1). After the improvement of vertigo's attack, usually 1-2 months, the volume of water intake is appropriately decreased, for example 1.2-1.5 day (20-25 [ml/kg/day] in F2). Most of patients with Meniere's disease have improved without re-attack for 1-2 years. They seem to be nervous and live under the very strong psychological pressure.

Initial Stage:
Water intake [ml/day/person]=30-35 [ml/kg/day]×body weight [kg] (F 1)
Second Stage:
Water intake [ml/day/person]=20-25 [ml/kg/day]×body weight [kg] (F2)
Possible Mechanism

1. Sufficient water intake during the short-term administration of osmotic diuretics (isosorbide®) improves endolymphatic hydrops and maintains plasma osmotic level low within the normal range.

2. The low plasma osmolality minimizes secretion of AVP from the pituitary gland.

3. Lower AVP levels in serum improve 1) water permeability in the inner ear, including Reissner's membrane, 2) production of endolymph through NKCC2 in stria vascularis, 3) microcirculation in the local arteries, including stria vascularis.

4. These actions together decrease endolymphatic pressure and prevent vertigo, hearing loss, and tinnitus.

An object of the present invention is to prevent vertiginous attack and hearing loss by maintaining low level of plasma AVP as a result of compliance instruction for proper intake of water to the patient with Meniere's disease.

### BRIEF EXPLANATION OF DRAWING

FIG. 1-FIG. 4 are the audiogram showing changes of pure tone audition of patients in clinical case 5.

### BEST MODE FOR CARRYING OUT THE INVENTION

The present invention is explained in detail by mentioning clinical cases.

Clinical Case 1

Meniere's Disease (Right)

The patient, male, 48 years old, body weight 68 kg, recognized subjective rotatory vertigo accompanied by aural fullness of the right ear from July, 2000. Thereafter, onset of rotatory vertigo accompanied by sensorineural hearing loss of the right low tone was repeated with the frequencies of once in 3 months. Treatment using hyperosmotic diuretic (isosorbide) and steroid was performed in each time of vertiginous attack and/or low frequency sensorineural hearing loss of the right ear.

Rotatory vertigo accompanied by aural fullness of the right ear appeared from Nov. 21, 2002, and the patient received the initial examination in Nov. 22, 2002 by Dr. Hideaki Naganuma, one of the inventors of the present invention, who belongs to the Dept. of Otolaryngology, the Kitasato Univ. Hospital, in Sagamihara-city, Kanagawa-pref., Japan. As a result, low frequency sensorineural hearing loss of the right ear and horizontal rotatory nystagmus to the left side were recognized. Treatment by administering isosorbide (63 g,t.d.s.)was performed for one week to improve vertigo and to remit aural fullness of the ear. At the time of reexamination performed in Dec. 6, 2002, the right ear hearing was improved to the normal level. Water intake, 2 lit. /day was initiated from that day. Thereafter in Jan. 24, 2003, the patient recognized subjective low frequency sensorineural hearing loss of the right ear and rotatory vertigo accompanied by the aural fullness of the right ear. The water intake was continued and isosorbide was administered (63 g, t.d.s.). Rotatory vertigo was improved within 3 hours, and the aural fullness in the right ear was also improved. At the time of reexamination performed in Feb. 7, 2003, low frequency sensorineural hearing loss of the right ear was improved, and the right ear hearing was within normal range. Thereafter, water intake was continued, and no rotatory vertigo was noted as well as progressing the right hearing within normal range up to March 2004.

The pure tone audiogram threshold of the right ear of the patient before the treatment (in Nov. 22, 2002) and after the treatment (in Aug., 29, 2003) at 125 Hz, 250 Hz, 500 Hz, 1000 Hz, 2000 Hz, 4000 Hz and 8000 Hz and pure-tone average at 500 Hz, 1000 Hz, 2000 Hz and 4000 Hz were shown in Table 1.

TABLE 1
The hearing levels of the right ear before and after
treatment

| | hearing level before | hearing level after |
|---|---|---|
| | treatment (dB) | treatment (dB) |
| 125 Hz | 45 | 0 |
| 250 Hz | 45 | 5 |
| 500 Hz | 45 | 15 |
| 1000 Hz | 50 | 15 |
| 2000 Hz | 35 | 15 |
| 4000 Hz | 10 | 10 |
| 8000 Hz | 15 | 25 |
| average | 35 | 13.75 |
| (500-4000 Hz) | | |

Clinical Case 2
Meniere's Disease (Bilateral)

The patient, female, 62 years old, body weight 65 kg, was repeated rotatory vertigo accompanied by aural fullness of bilateral ears. From August 2000 to August 2001, aural fullness of bilateral ears and onset of rotatory vertigo accompanied by resound in the right ear were recognized with the frequencies of once in about one month. The patient recognized bilateral tinnitus every day. Duration of rotatory vertigo in one time was about 3 hours.

From September 2001 to November 2002, frequency of onset of similar rotatory vertigo was increased to once in about 10 days. Treatment was continued for that time by another doctor. In Nov. 19, 2002, the patient received the initial diagnosis in this department. Although the pure tone audiogram threshold of the bilateral ear was within normal range, horizontal nystagmus for left direction was recognized. Treatment with administering isosorbide (21 g/dose) and difenidol hydrochloride (25 mg/dose) (which were orally administered when rotatory vertigo, hearing loss or aural fullness were recognized) was initiated. Thereafter, rotatory vertigo was recognized in Dec. 10, 2002, Dec. 21, 2002, Jan. 10, 2003, January 13, January 29, and February 12, and in January 19 and 20, resound in the right ear was recognized.

From Feb. 21, 2003, in addition to the treatment with administering isosorbide (21 g/dose) and difenidol hydrochloride (25 mg/dose) (which were orally administered when rotatory vertigo, hearing loss or aural fullness were recognized), the treatment with water intake, 1.5 lit./day, was performed. Thereafter, rotatory vertigo was recognized in Mar. 2, 2003, Mar. 11, 2003, Mar. 12, 2003 and May 3, and resound or aural fullness (in the right ear) was recognized in Mar. 19, 2003, March 20, April 2, April 3, April 26, May 10, July 7, July 8, July 24, August 8 and August 18.

Rotatory vertigo was remitted after May 3, 2003, and resound or aural fullness (in the right ear) was remitted after Aug. 18, 2003, and the hearing level was maintained within normal range to February 2004.

Clinical Case 3

Meniere's Disease (Left)

The patient, male, 28 years old, body weight 70 kg, recognized suffering from rotatory vertigo in Aug. 10, 1999. Thereafter onset of rotatory vertigo was recognized with a frequency of once in 2 months.

The patient recognized rotatory vertigo accompanied by left tinnitus in Mar. 5, 2000 and visited this Department in Mar. 6, 2000 for receiving initial examination. Horizontal rotatory nystagmus to the left side and low frequency sensorineural hearing loss of the left ear were recognized. Vertigo was remitted, and isosorbide (63 g, t.d.s.) and difenidol hydrochloride (75 mg, t.d.s.) were administered for 14 days, and the left hearing was improved in March 31. During the follow-up period, rotatory vertigo accompanied by low and high frequency sensorineural hearing loss of the left ear was recognized from Jul. 22, 2000, and isosorbide (63 g, t.d.s.) and difenidol hydrochloride (75 mg, t.d.s.) were administered for 6 days, and the left hearing was improved to normal range in July 28. Water intake, 300 ml, before going to sleep was initiated from Aug. 5, 2000 (after December 2000, water intake was changed to 2 lit./day). In Aug. 15, 2000, hearing loss of the left ear and left ear tinnitus (high frequency) were recognized, and the reexamination was performed in Aug. 16, 2000. Treatment using steroid was performed due to recognizing high frequency sensorineural hearing loss and the left hearing was improved within normal range in August 26.

Thereafter, no rotatory vertigo was recognized up to the present day (January 2004) except for recognizing rotatory vertigo accompanied by low frequency sensorineural hearing loss of the left ear in Mar. 16, 2001, and the hearing was maintained within normal range.

In the present case who could be followed up for more than 24 months after treatment with water intake, the therapeutic effect was judged by using the criteria of AAO-HNS (1995)^{14), 15)}. Proviso that in an examination on changes of the hearing, although the criteria in the four-frequency pure-tone averages (PTA) of AAO-HNS (1995)¹⁴⁾ adopted mean values of the hearing levels consisting of 500 Hz, 1000 Hz, 2000 Hz and 3000 Hz, in our case, the value of 4000 Hz was used in stead of that of 3000 Hz. Table 2 shows the poorest hearing levels of the left ear during 6 months before the treatment and those during 18-24 months after treatment.

TABLE 2
The hearing levels of the left ear before and after
treatment

| | hearing level before | hearing level after |
|---|---|---|
| | treatment (dB) | treatment (dB) |
| | (Jul. 24, 2000) | (Jun. 7, 2002) |
| 125 Hz | 40 | 25 |
| 250 Hz | 25 | 20 |
| 500 Hz | 25 | 15 |
| 1000 Hz | 15 | 5 |
| 2000 Hz | 10 | 5 |
| 4000 Hz | 45 | 5 |
| 8000 Hz | 80 | 15 |
| average | 23.75 | 7.5 |
| (500-4000 Hz) | | |

In the present case, the hearing after the treatment was improved in 16.25 dB as compared with before the treatment, and this can be judged as a category within “improved”.

Therapeutic effect for vertigo^{14), 15)} was calculated according to the following equation.

$$\frac{\begin{matrix}the average number of definitive spells per month \\ for the 6 months 18 to 24 months after therapy\end{matrix}}{\begin{matrix}the average number of definitive spells per month \\ for the 6 months before therapy\end{matrix}} \times 100$$

Namely, in the present case, vertiginous attack in 6 months during 18 months-24 months after the treatment was 0 time, and average numbers of rotatory vertigo per month for 6 months before the treatment were 2 times (i.e. onset in Mar. 5, 2000 and Jul. 22, 2000 within 6 months before Aug. 5, 2000). Consequently, applying the above equation:

$$\frac{0 / 6}{2 / 6} \times 100 = 0$$

This result is judged to be included in a category of “complete control”.
Clinical Case 4
Low Frequency Sensorineural Hearing Loss of the Right Ear

The patient, female, 75 years old, body weight 55 kg, recognized hearing loss of the right ear. No vertigo was noted. Low frequency sensorineural hearing loss of the right ear was recognized by another doctor in Jun. 2, 2003

The patient received initial examination in this department in Jun. 3, 2003. Low frequency sensorineural hearing loss of the right ear and bilateral sensorineural hearing loss in the high frequency (2000 Hz -8000 Hz) which might be caused by aging were recognized. The right tinnitus was recognized subjectively from May 2003. Though isosorbide (63 g, t.d.s.) was administered orally for 2 weeks, no improvement in the right hearing could be obtained. Water intake (1.5 lit./day) was performed and followed up from Jun. 20, 2003. Thereafter, the hearing of the right low tone was improved in August 2003, but the hearing of the right low tone was decreased again in September 2003. The hearing of the right low tone was improved again in October 2003, and the hearing of the right low tone was maintained improved state to the present day (January 2004). Progress of the hearing level (dB) from the initial examination in Jun. 23, 2003 to January 2004 is shown in Table 3.

TABLE 3
Progress in treatment of hearing level from initiation
of water intake therapy

| | 2003 | | | | | | | 2004 |
|---|---|---|---|---|---|---|---|---|
| | Jun. | Jul. | Aug. | Sep. | Oct. | Nov. | Dec. | Jan. |
| 125 Hz | 30 | 50 | 25 | 30 | 30 | 25 | 25 | 25 |
| 250 Hz | 35 | 45 | 25 | 40 | 30 | 20 | 25 | 25 |
| 500 Hz | 40 | 45 | 25 | 40 | 25 | 20 | 15 | 20 |
| 1000 Hz | 50 | 50 | 30 | 45 | 25 | 30 | 25 | 25 |
| 2000 Hz | 25 | 45 | 20 | 40 | 20 | 20 | 20 | 20 |
| 4000 Hz | 25 | 60 | 30 | 40 | 30 | 35 | 30 | 25 |
| 8000 Hz | 75 | 85 | 75 | 80 | 80 | 65 | 60 | 65 |
| average | 35.0 | 50.0 | 26.25 | 41.25 | 25.0 | 23.75 | 22.5 | 22.5 |
| (500-4000 Hz) | | | | | | | | |

Clinical Case 5
Meniere's Disease (Left)

The patient, female 51 years old, body weight 55 kg, recognized aural fullness of the left ear and hearing loss of the left ear. Although steroid was administered by the doctor from September 29, hearing level was further reduced and received the initial examination in this department in Oct. 4, 2000.

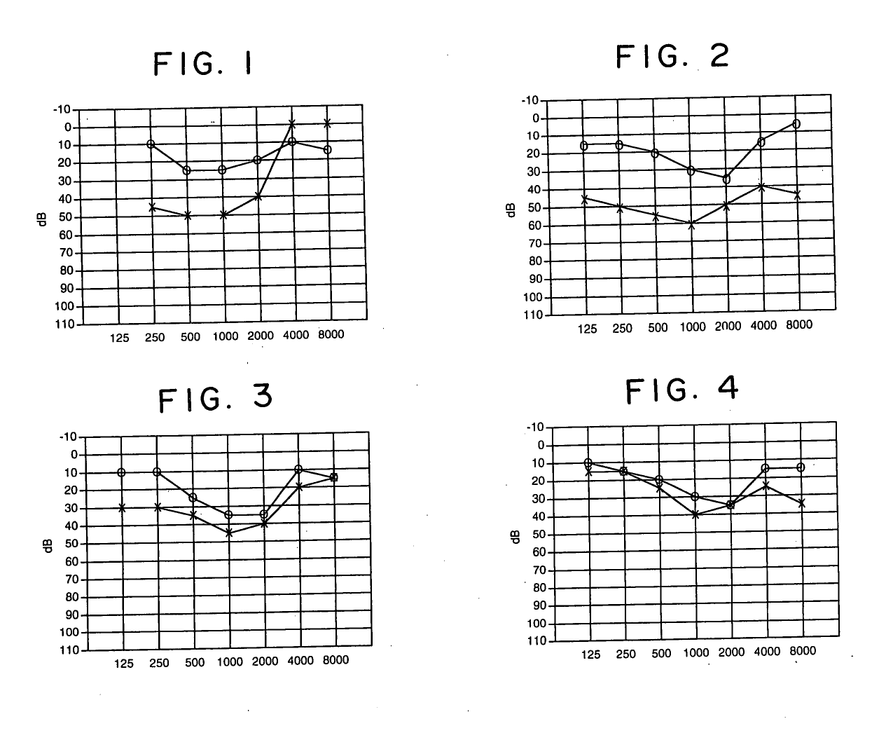

In the initial examination (Oct. 4, 2000), low frequency sensorineural hearing loss of the left ear was recognized and the patient was admitted to the hospital (refer to FIG. 1 ). Although treatment using steroid, VB12 and vasodilator (prostaglandin:PGE₁) was continued, no improvement in low frequency sensorineural hearing loss of the left ear was recognized with enhanced left ear tinnitus, and the progress was followed up. The left ear tinnitus was further enhanced in Aug. 19, 2002, and rotatory vertigo was subjectively recognized in August 23, and the low frequency hearing level was aggravated. In addition to sensorineural hearing loss of the left low tone, the hearing of the left high tone was decreased (sensorineural hearing loss), and rotatory vertigo was again recognized in Sep. 6, 2002 (refer to FIG. 2). In addition to oral administration of isosorbide (63 g, t.d.s.) and vasodilator (Adenosine triphosphate (300 mg, t.d.s)) for 2 weeks, water intake (1.5 lit./day) was initiated and the progress was followed up. High frequency sensorineural hearing loss of the left ear was improved in Oct. 24, 2002. Thereafter low frequency sensorineural hearing loss of the left ear was improved from Nov. 29, 2002 (refer to FIG. 3), and the left hearing was gradually equalized with the right hearing, and the left hearing became equal level to the right hearing from Jul. 18, 2003 (refer to FIG. 4). Further, rotatory vertigo could not be recognized after Oct. 24, 2002, and the left ear tinnitus was gradually remitted to January 2004.

Audiogram in FIG. 1-FIG. 4 shows changes in the pure tone hearing level. In each figure, a horizontal axis indicates frequency (Hz) and a vertical axis indicates hearing level. In the figure, the hearing level in each frequency of the right ear (o-o) and the left ear (x-x) is shown. A graph in the upper position indicates better hearing level.

As obvious from referring to FIG. 1, in the initial examination in Oct. 4, 2000, hearing loss of about 50 dB in the left low tone was recognized. The bone conduction value was measured in the actual pure tone hearing test, though not shown in FIG. 1, and the hearing loss was confirmed to be sensorineural hearing loss. The hearing loss of the left ear was continued thereafter, although it was slightly fluctuated. Although the hearing level of the other side (right ear) was almost normal in the initial examination, the hearing level of the right ear deteriorated gradually at 500 Hz-2000 Hz. As obvious by referring to FIG. 2, the hearing level of the left ear deteriorated in the high tone range in addition to the low tone range depending on the rotatory vertigo in Sep. 6, 2002. Consequently, as a result of same dosing as in the past (isosorbide (63 g, t.d.s), vasodilator, 63 g t.i.d, Adenosine triphosphate (300 mg, t.d.s)) for 2 weeks and initiating water intake therapy (1.5. lit./day), as obvious from referring to FIG. 3, the hearing level of the left ear began to improve from Nov. 29, 2002. As obvious from FIG. 4, the hearing of the left ear was almost reached to same level of the hearing of the right ear.

Clinical Case 6

Meniere's Disease (Right)

The patient, female, 60 years old, body weight 58 kg, recognized subjectively rotatory vertigo accompanied by the right hearing loss with a frequency of once in 3 months from March 1985. Frequency of the similar vertiginous attack was reduced to twice in a year from 1993. However, the frequency of the similar vertiginous attack was increased again to once in two months from 2001. In the morning of Jul. 8, 2001, rotatory vertigo accompanied by right ear tinnitus was recognized and was continued for 6 hours. The patient was treated by long term administration of isosorbide from 1985 in another hospital.

In Jul. 11, 2001, the patient received initial examination in this department. Horizontal sensorineural hearing loss with about 85 dB was recognized in the right ear. In Aug. 2, 2001, rotatory vertigo accompanied by increased right ear tinnitus was subjectively recognized. Althoughnolargevariation was recognized in the right hearing level, horizontal rotatory nystagmus to the left side was recognized. Treatment with administering isosorbide (21 g/dose) and difenidol hydrochloride (25 mg/dose) (which were orally administered when rotatory vertigo, hearing loss or aural fullness were recognized) was initiated. Since rotatory vertigo was also subjectively recognized in August 5, in addition to the treatment with administering isosorbide (21 g/dose) and difenidol hydrochloride (25 mg/dose) (which were orally administered when rotatory vertigo, hearing loss or aural fullness were recognized), water intake 300 ml before going to sleep was initiated from August 10 (after Nov. 12, 2002, water intake was changed to 1.5 lit./day). Thereafter the therapy with administering isosorbide (21 g/dose) and difenidol hydrochloride (25 mg/dose) (which were orally administered when rotatory vertigo, hearing loss or aural fullness were recognized) and water intake (1.5 lit./day) were continued. Although rotatory vertigo accompanied by enhanced right ear tinnitus was recognized in Aug. 21, 2001, August 22, September 12, November 3, November 15, November 18, November 20, November22, December 1, December 5 and December21, thereafter no rotary vertiginous attack accompanied by left ear tinnitus was noted until now (February 2004).

In the present case who could be followed up for more than 24 months after treatment with water intake, the therapeutic effect was judged by using the criteria of AAO-HNS (1995)^{14), 15)}. Proviso that in an examination on changes of the hearing, although the criteria in the four-frequency pure-tone averages (PTA) of AAO-HNS (1995)¹⁴⁾ adopted mean values of the hearing levels consisting of 500 Hz, 1000 Hz, 2000 Hz and 3000 Hz, in our case, the value of 4000 Hz was used in stead of that of 3000 Hz. Table 4 shows the poorest hearing levels of the right ear during 6 months before the treatment and those during 18-24 months after treatment.

TABLE 4
The hearing levels of the right ear before and after
treatment

| | hearing level before | hearing level after |
|---|---|---|
| | treatment (dB) | treatment (dB) |
| | (Aug. 3, 2001) | (Apr. 11, 2002) |
| 125 Hz | — | 65 |
| 250 Hz | 85 | 80 |
| 500 Hz | 85 | 80 |
| 1000 Hz | 85 | 85 |
| 2000 Hz | 75 | 75 |
| 4000 Hz | 90 | 90 |
| 8000 Hz | 95 | 90 |
| average | 83.75 | 82.5 |
| (500-4000 Hz) | | |

In the present case, the hearing was improved in 1.25 dB as compared with before the treatment, and this can be judged as a category in “unchanged”.

Therapeutic effect for vertigo ^{14), 15)} was calculated according to the following equation.

$$\frac{\begin{matrix}the average number of definitive spells per month \\ for the 6 months 18 to 24 months after therapy\end{matrix}}{\begin{matrix}the average number of definitive spells per month \\ for the 6 months before therapy\end{matrix}} \times 100$$

Namely, in the present case, vertiginous attack in 6 months during 18 months-24 months after the treatment was 0 time, and the attack occurred in August 5 and July 8 before the treatment, and assuming that rotatory vertigo occurred in a frequency of once in 2 months before these date, vertiginous attack might occur in March and May. Namely, totally 4 times of onset of rotatory vertigo might be estimated during 6 months from February 10 to Aug. 10, 2001. Consequently, applying the above equation:

$$\frac{0 / 6}{4 / 6} \times 100 = 0$$

This result is judged to be included in a category of “complete control”.
Clinical Case 7
Meniere's Disease (Left)

The patient, female, 30 years old, body weight 50 kg, recognized bilateral aural fullness and hearing loss of the left ear from Mar. 1, 2002. No vertigo was noted. In Mar. 5, 2002, (low and high frequency) sensorineural hearing loss of the left ear was recognized in the other hospital, and the patient received the examination in this department in Mar. 6, 2002. Low and high frequency sensorineural hearing loss of the left ear was also recognized and the patient was hospitalized. In addition to the treatment with steroid, VB12 and vasodilator (prostaglandin:PGE₁), water intake 300 ml before going to sleep was initiated. As a result, low frequency sensorineural hearing loss of the left ear was improved. The patient compliance instruction for water intake was performed continuously in the outpatient department (after September 2002, water intake was changed to 1.5 lit. /day), and the left hearing could be maintained the improved state until now.

In the present case who could be followed up for more than 24 months after treatment with water intake, the therapeutic effect was judged by using the criteria of AAO-HNS (1995) ^{14), 15)}. Proviso that in an examination on changes of the hearing, although the criteria in the four-frequency pure-tone averages (PTA) of AAO-HNS (1995)¹⁴⁾ adopted mean values of the hearing levels consisting of 500 Hz, 1000 Hz, 2000 Hz and 3000 Hz, in our case, the value of 4000 Hz was used in stead of that of 3000 Hz. Table 5 shows the poorest hearing levels of the left ear during 6 months before the treatment and those during 18-24 months after treatment.

TABLE 5
The hearing levels of the left ear before and after
treatment

| | hearing level before | hearing level after |
|---|---|---|
| | treatment (dB) | treatment (dB) |
| | (Mar. 6, 2002) | (Oct. 10, 2003) |
| 125 Hz | 35 | 5 |
| 250 Hz | 40 | 5 |
| 500 Hz | 30 | 5 |
| 1000 Hz | 20 | 5 |
| 2000 Hz | 10 | 5 |
| 4000 Hz | 30 | 20 |
| 8000 Hz | 40 | 45 |
| average | 22.5 | 8.75 |
| (500-4000 Hz) | | |

In the present case, hearing was improved in 13.75 dB as compared with before the treatment, and this can be judged as a category in “improved”.

Therapeutic effect for vertigo^{14), 15)} was tried to calculate according to the following equation.

$$\frac{\begin{matrix}the average number of definitive spells per month \\ for the 6 months 18 to 24 months after therapy\end{matrix}}{\begin{matrix}the average number of definitive spells per month \\ for the 6 months before therapy\end{matrix}} \times 100$$

However, in the present case, vertiginous attack in 6 months during 18 months-24 months after the treatment was 0 time, and rotatory vertigo occurred during 6 months before the treatment was also 0 time. Consequently, the denominator is 0, indicating impossible to calculate, however since the numerator is also 0, this can be judged to be included in a category of “complete control”.

Clinical Case 8

Meniere's Disease (Right)

The patient, female, 66 years old, body weight 44 kg, recognized subjectively rotatory vertigo accompanied by the right hearing loss in May 2001. In November 2001, similar vertigo was recognized, and the patient received long term consecutive administration of isosorbide in the other hospital. However, the patient received initial examination in this department in Jan. 9, 2002 due to decreased right hearing. In the initial examination, the right hearing exhibited sensorineural hearing loss with lower value at 2 -25 dB in all frequencies as compared with the left hearing. Since the patient was administered orally isosorbide consecutively for long term, the consecutive administration was terminated, and the compliance instruction for administering isosorbide (21 g/dose) (which was orally administered when rotatory vertigo, hearing loss or aural fullness were recognized) was performed. In addition, water intake (300 ml before going to sleep; after Nov. 22, 2002, water intake was changed to 1.5 lit./day) was initiated from Feb. 22, 2002. As a result, although the hearing level of the right low tone range was decreased from Feb. 22, 2002 to May 10, 2002, the hearing level of the right low tone range was gradually improved after Jun. 7, 2002, and the right hearing level was reached to the low level of 5 -15 dB as compared with the left hearing level in all frequency range at present. No rotatory vertigo could be recognized during the progress after the initial examination.

In the present case who could be followed up for more than 24 months after treatment with water intake, the therapeutic effect was judged by using the criteria of AAO-HNS (1995) ^{14), 15)}. Proviso that in an examination on changes of the hearing, although the criteria in the four-frequency pure-tone averages (PTA) of AAO-HNS (1995)¹⁴⁾ adopted mean values of the hearing levels consisting of 500 Hz, 1000 Hz, 2000 Hz and 3000 Hz, in our case, the value of 4000 Hz was used in stead of that of 3000 Hz. Table 6 shows the poorest hearing levels of the right ear during 6 months before the treatment and those during 18-24 months after treatment.

TABLE 6
The hearing levels of the right ear before and after
treatment

| | hearing level before | hearing level after |
|---|---|---|
| | treatment (dB) | treatment (dB) |
| | (Feb. 22, 2002) | (Sep. 19, 2003) |
| 125 Hz | 60 | 40 |
| 250 Hz | 70 | 40 |
| 500 Hz | 60 | 35 |
| 1000 Hz | 60 | 40 |
| 2000 Hz | 50 | 40 |
| 4000 Hz | 65 | 60 |
| 8000 Hz | 85 | 80 |
| average | 58.75 | 43.75 |
| (500-4000 Hz) | | |

In the present case, the hearing was improved in 15.0 dB as compared with before the treatment, and this can be judged as a category on “improved”.

Therapeutic effect for vertigo ^{14), 15)} was calculated according to the following equation.

$$\frac{\begin{matrix}the average number of definitive spells per month \\ for the 6 months 18 to 24 months after therapy\end{matrix}}{\begin{matrix}the average number of definitive spells per month \\ for the 6 months before therapy\end{matrix}} \times 100$$

Namely, in the present case, vertiginous attack in 6 months during 18 months -24 months after the treatment was 0 time, and the number of times of rotatory vertigo occurred in 6 months before the treatment was 1 (November 2001). Consequently, applying the above equation:

$$\frac{0 / 6}{4 / 6} \times 100 = 0$$

This result is judged to be included in a category of “complete control”.

### INDUSTRIAL APPLICABILITY

After confirming no abnormality of the circulatory system and the kidney system in the patient, considering body weight, age and lifestyle, performing water intake necessary for one day and short term administration of osmotic diuretic, maintaining plasma osmotic pressure at the lowest limit of the normal level, minimizing the secretion of AVP from the pituitary grand, decreasing influent of water into the endolymph, and preventing vertiginous attack as well as improving hearing. Accordingly, the present invention is an epoch-making therapeutic method for Meniere's disease and endolymphatic hydrops associated diseases.

### REFERENCES

¹⁾:Beitz, e., Kumagami, H., Krippeit-Drews, P., Ruppersberg, J. P.,Schultz, J. E., 1999. Expression pattern of aquaporin water channels in the inner ear of the rat. The molecular basis for awater regulation system in the endolymphatic sac. Hear. Res. 132, 76-84.
²⁾:Fukusima, M., Kitahara, T., Uno, Y., Fuse, Y., Doi, K., Kubo, T., 2002. Effects of intratympanic injection of steroids on changes in rat inner ear aquaporin expression. Acta Otolaryngol, 122, 600-606.
³⁾:Huang, D., Chen, S., Nagura, M., Lim, D. J., Lin, X., 2002. Expression patterns of aquaporins in the inner ear: evidence for concerted actions of multiple of aquaporins to facilitate water transport in the cochlea. Hear. Res. 165, 85-95
⁴⁾:Kumagami, H., Oewenheim, H., Beitz, E., Wild, K., Schwartz, H., Yamashita, K., Schultz, J., Paysan, J., Zenner, H. P., Ruppersberg, J. P., 1998. The effect of anti-diuretic hormone on the endolymphatic sac of the inner ear. Pflugers Arch. 436, 970-975.
⁵⁾:Mhatre, A. N., Steibach, S., Hribar, K., Hoque, A. T., Lalwani, A. K., 1999. Identification of aquaporin 5(AQP5) within the cochlea: cDNA cloning and in site localization. Biochem. Biophys. Res. Commun.264, 157-162.
⁶⁾:Mhatre, A., Jero, J., Chiappini, I., Bolasco, G., Barbara, M., Lalwani, A., 2002. Aquaporin-2expressioninthemammalian cochlea and investigation of its role in Meniere's disease. Hear. Res. 170, 59-69.
⁷⁾:Minami, Y., Shimada, S., Miyahara, H., Matsunaga, T., Toyama, M., 1998. Selective expression of mercurial-insensitive water channel (AQP-4) gene in Hensen and Claudius Cells In the rat cochlea. Acta Otolayngol. (Stock.) 533 (Suppl.) 19-21.
⁸⁾:Sawada, S., Takeda, T., Kitano, H., Takeuchi, S., Kakigi, A., Azuma, M., 2002. Aquaporin-1 regulation by vasopressin in the rat inner ear. Neuro Report 13, 1127-1129.
⁹⁾:Stankovic, K, M., Adams, J. C., Brown, D., 1995. Immunolocalization of aquaporin CHIP in the guinea pig inner ear. Am. J. Physiol. 269, C1450-C1456.
¹⁰⁾:Takumi, Y., Nagelhus, E, A., Eidet, J., Matsubara, A., Usami, S., Shinkawa, H., Nielsen, Sl, Ottersen, 0, P., 1998. Select types of supporting cell in the inner ear express aquaporin-4 water channel protein. Eur. J. Neurosci. 10, 3584-3595.
¹¹⁾:Takeda, T., Takeda, S., Kitano, H., Okada, S., Kakigi, A., 2000. Endolymphatic hydrops induced by chronic administrayion vasopressin. Hear. Res. 140, 1-6.
¹²⁾:Takeda, T., Kakigi, A., Saito, H., 1995. Antidiuretichormone (ADH) and endolymphatic hydrops. Acta. Otolaryngol. (Stockh.) 519 (Suppl.) 219-222.
¹³⁾:Moffat, D. A., Ballagh, R. H. Meniere's disease. In Scott-Brown's Otolaryngology (6^{th} ed) Otology, ed. kerr, A. G. Butter worth-Heinemann, Oxford, 1997, pages 3/19/1-3/19/50.
¹⁴⁾: Committee on Hearing and Equilibrium: Committee on Hearing and Equilibrium guidelines for the diagnosis and evaluation of therapy in Meniere's disease. Otolaryngol Head Neck Surg 113: 181-185, 1995.
¹⁵⁾: Committee on Hearing and Equilibrium: Guidelines for reporting treatment results in Meniere's disease. Otolaryngol Head Neck Surg 93: 579-581, 1985.
¹⁶⁾:Preston., G. M., Carro II, T. P., Guggino, W.B., AGRE, P. Appearance of water channels in Xenopus oocytes expressing red cell CHIP28 protein, Science, 256 (5055): 385-387, 1992.
¹⁷⁾:Fushimi, K., Uchida, S., Haea, Y., Hirata, Y., Marumo, F., Sasaki, S. Cloning and expression of apical membrane water channel of rat kidney collecting tubule. Nature 361 (6412): 549-552, 1993.
¹⁸⁾:Naganuma, H., Kawahara, K. et al. Effects of arginine-vasopressin on auditory brainstem evoked response (ABR) threshold in rats. Otol Jpn 12(4):402, 2002.
¹⁹⁾:Harrison M. S., Naftalin L. Meniere's disease: mechanism and magagement. Springfield (IL): Charles C. Thomas; 1968.
²⁰⁾:Lim, J. S., Lange, M. E. et al. Serum antiduretic hormone levels in patients with unilateral Meniere's disease Laryngoscope 113: 1321-1326, 2003.

What is claimed is:

1. A therapeutic method for endolymphatic hydrops associated diseases comprising after confirming no abnormality of the circulatory system and the kidney system in the patient, considering body weight, age and lifestyle, and performing compliance instruction of water intake necessary for one day in the patient.

2. The therapeutic method for endolymphatic hydrops associated diseases according to claim 1 comprising performing water intake necessary for one day depending on the symptom, maintaining plasma osmotic pressure at the lowest limit of the normal level, minimizing the secretion of AVP from the pituitary grand, and decreasing inf luent of water into the endolymph to prevent vertiginous attack and improve hearing..
